(19) United States
(12) Patent Application Publication
(10) Pub. No.: US 2026/0047990 A1
(43) Pub. Date: Feb. 19, 2026

(54) Compositions and Methods for Treating Skin Discoloration

(71) Applicant: Racquel Frisella, LLC, Kirkwood, MO (US)

(72) Inventors: Racquel Catherine Frisella, Kirkwood, MO (US); Steven Hernandez, Blue Point, NY (US)

(21) Appl. No.: 19/298,831

(22) Filed: Aug. 13, 2025

Related U.S. Application Data
(60) Provisional application No. 63/683,021, filed on Aug. 14, 2024.

(51) Int. Cl. A61K 8/49; A61K 8/19; A61K 8/34; A61K 8/60; A61Q 19/02

(52) U.S. Cl. CPC A61K 8/498; A61K 8/19; A61K 8/347; A61K 8/602; A61Q 19/02

(57) ABSTRACT

Disclosed in certain embodiments herein is a topical formulation including a tyrosine inhibitor, a skin brightener, and a resorcinol compound. The topical formulation does not include hydroquinone to comply with new federal regulations. A delivery system and method of treating hyperpigmentation is also provided.

### CROSS REFERENCE TO RELATED APPLICATION(S)

The present application claims priority to U.S. Provisional Application No. 63/683,021 filed on Aug. 14, 2024. The entire contents of which are incorporated in its entirety.

### FIELD OF THE INVENTION

The present invention relates to cosmetic compositions for skin care applications to treat skin discoloration, melasma, or hyperpigmentation. In particular, the cosmetic compositions includes a tyrosine inhibitor, a skin brightener, and a resorcinol compound.

### BACKGROUND OF THE INVENTION

Hyperpigmentation refers to the darkening of pigment on the skin because of sun damage or melasma and is common for many people. Hyperpigmentation often appears in the form of dark spots, sun sports or melasma. Thus, any darkening of the skin may cause an uneven complexion.

Most cosmetic formulations on the market to treat hyperpigmentation contain hydroquinone, which cause irritation. Though hydroquinone is effective at treating hyperpigmentation, regulations have been placed to avoid users experiencing irritation. For example, cosmetic formulations including hydroquinone are only available to those with a prescription. Moreover, traditional treatment of hyperpigmentation using a prescription formulation including hydroquinone requires a long treatment time to show improvement and has a variety of adverse effects, such as irritation.

Therefore, there is a need for a topical formulation that treats hyperpigmentation without the use of hydroquinone and does not cause irritation.

### SUMMARY

It is an object of certain embodiments of the disclosure to provide a topical formulation, and a method of using the topical formulation for improving the pigmentation of the skin. It has been found that combining a tyrosine inhibitor, a skin brightener and a resorcinol compound was found to have similar or even better results than a traditional formulation including hydroquinone.

In an embodiment, a topical formulation is provided. The topical formulation includes a tyrosine inhibitor, a skin brightener, and a resorcinol compound.

In certain embodiments, the tyrosine inhibitor may include kojic acid.

In certain embodiments, the skin brightener may include a glycosylated derivative of hydroquinone. In some embodiments, the glycosylated derivative of hydroquinone may include arbutin.

In certain embodiments, the resorcinol compound may include an alkyl resorcinol, an aromatic resorcinol or a combination thereof. In some embodiments, the alkyl resorcinol may include at least one C1-C8 resorcinol. In some embodiments, the aromatic resorcinol may include a phenyl alkyl resorcinol. In some embodiments, the resorcinol compound may include hexyl resorcinol, butyl resorcinol, phenylethyl resorcinol, or a combination thereof.

In certain embodiments, the topical formulation does not include hydroquinone.

In certain embodiments, the tyrosine inhibitor may be included in an amount of about 0.5 wt % to about 10 wt % based on total weight of the formulation. In certain embodiments, the kojic acid may be included in an amount of about 0.5 wt % to about 10 wt %, based on total weight of the formulation.

In certain embodiments, the glycosylated derivative of hydroquinone may be included in an amount of about 1 wt % to about 10 wt % based on total weight of the formulation. In certain embodiments, the arbutin may be included in an amount of about 1 wt % to about 10 wt % based on total weight of the formulation.

In certain embodiments, the resorcinol compound may include hexyl resorcinol in an amount of about 0.1 wt % to about 10 wt %, based on total weight of the formulation. In certain embodiments, the resorcinol compound may include butyl resorcinol in an amount of 0.1 wt % to about 10 wt %, based on total weight of the formulation. In certain embodiments, the resorcinol compound may include phenylethyl resorcinol in an amount of about 0.01 wt % to about 10 wt %, based on total weight of the formulation.

In certain embodiments, the resorcinol compound may include hexyl resorcinol in an amount of about 0.1 wt % to about 10 wt %, butyl resorcinol in an amount of about 0.1 wt % to about 10 wt %, and phenylethyl resorcinol in an amount of about 0.01 wt % to about 10 wt %, based on total weight of the formulation.

In certain embodiments, the topical formulation may further include an ascorbate component. In some embodiments, the ascorbate component may include ascorbic acid, derivatives thereof or combinations thereof. In some embodiments, the ascorbate component may include ascorbic acid, ascorbyl palmitate, sodium ascorbate, potassium ascorbate, ammonium ascorbate, triethanolamine ascorbate, ascorbyl phosphate or magnesium ascorbyl phosphate, ascorbic acid polypeptides, ascorbyl glucosamine, ascorbic acid polymers, esters of ascorbic acid, amides of ascorbic acid, L-ascorbic acid, tetrahexyldecyl ascorbate or combinations thereof.

In certain embodiments, the topical formulation may further include a solvent. In some embodiments, the solvent may include water, ethyoxydiglycol, alcohol, alkanediol, glycerin, an aromatic alcohol, or a combination thereof.

In certain embodiments, water may be included in an amount of about 20 wt % to about 80 wt %, based on total weight of the formulation.

In certain embodiments, the ethoxydiglycol may be included in an amount of about 1 wt % to about 25 wt %, based on total weight of the formulation.

In certain embodiments, the alcohol may include ethanol. In some embodiments, the alcohol may be included in an amount of about 1 wt % to about 50 wt %, based on total weight of the formulation.

In certain embodiments, the alkanediol may include ethylene glycol, propylene glycol, or butylene glycol. In some embodiments, the alkanediol may include butylene glycol. In some embodiments, butylene glycol may be included in an amount of about 1 wt % to about 30 wt %, based on total weight of the formulation. In some embodiments, the alkanediol may be included in an amount of about 1 wt % to about 30 wt %, based on total weight of the formulation.

In certain embodiments, glycerin may be included in an amount of about 0.001 wt % to about 20 wt %, based on total weight of the formulation.

In certain embodiments, the aromatic alcohol may include benzyl alcohol. In some embodiments, the benzyl alcohol may be included in an amount of about 0.001 wt % to about 10 wt %, based on total weight of the formulation. In some embodiments, the aromatic alcohol may be included in an amount of about 0.001 wt % to about 10 wt %, based on total weight of the formulation.

In certain embodiments, the topical formulation may further include a keratolytic. In some embodiments, the keratolytic may include salicylic acid, urea, lactic acid, glycolic acid, or a combination thereof. In some embodiments, the keratolytic may include salicylic acid. In some embodiments, the salicylic acid may be included in an amount of about 0.1 wt % to about 10 wt %, based on total weight of the formulation. In some embodiments, the keratolytic may be included in an amount of about 0.1 wt % to about 10 wt %, based on total weight of the formulation.

In certain embodiments, the topical formulation may further include an emulsifier. In some embodiments, the emulsifier may include polysorbate 20, polysorbate 40, polysorbate 60, polysorbate 80, potassium cetyl sulfate, lauryl alcohol and polyethylene glycol, or a combination thereof. In some embodiments, the emulsifier may include lauryl alcohol and polyethylene glycol. In some embodiments, the lauryl alcohol and polyethylene glycol may be included in an amount of about 0.01 wt % to about 10 wt %, based on total weight of the formulation. In some embodiments, the emulsifier may be included in an amount of about 0.01 wt % to about 10 wt %, based on total weight of the formulation.

In certain embodiments, the topical formulation may further include potassium hydroxide. In some embodiments, the potassium hydroxide may be included in an amount of about 0.0001 wt % to about 5 wt %, based on total weight of the formulation.

In certain embodiments, the topical formulation may further include a chelating agent. In some embodiments, the chelating agent may include disodium ethylenediaminetetraacetic acid (EDTA), diethylenetriaminepentaacetic acid (DTPA), N-(hydroxyethyl)-ethylenediaminetriacetic acid (HEDTA), nitrilotriacetic acid (NTA), or a combination thereof. In some embodiments, the chelating agent may include disodium EDTA. In some embodiments, the disodium EDTA may be included in an amount of about 0.0001 wt % to about 0.5 wt %, based on total weight of the formulation. In some embodiments, the chelating agent may be included in an amount of about 0.0001 wt % to about 0.5 wt %, based on total weight of the formulation.

In certain embodiments, the topical formulation may further include an antioxidant. In some embodiments, the antioxidant may include a hydroxy acid. In some embodiments, the hydroxy acid may include citric acid, lactic acid, malic acid, or a combination thereof. In some embodiments, the hydroxy acid may include citric acid. In some embodiments, the citric acid may be included in an amount of about 0.001 wt % to about 1 wt %, based on total weight of the formulation. In some embodiments, the antioxidant may be included in an amount of about 0.001 wt % to about 1 wt %, based on total weight of the formulation.

In certain embodiments, the topical formulation may further include a botanical extract. In some embodiments, the botanical extract may include thyme herb, mulberry extract, willow bark extract, elderberry, Echinacea, cucumber base extract, or a combination thereof. In some embodiments, the botanical extract may be included in an amount of about 0.001 wt % to about 10 wt %, based on total weight of the formulation.

In certain embodiments, the topical formulation may further include a polyphenol. In some embodiments, the polyphenol may include a green tea extract. In some embodiments, the green tea extract may include Camellia sinensis extract. In some embodiments, the Camellia sinensis extract may be included in an amount of about 0.001 wt % to about 10 wt %, based on total weight of the formulation.

In another embodiment, a delivery system is provided. The delivery system may include an applicator pad infused with a topical formulation of the present disclosure.

In yet another embodiment, a method of treating is provided. The method may include applying a topical formulation to the skin of a subject. The method may further include that applying may be performed with an applicator pad or an applicator device. In some embodiments, the applicator device may include a roll-on device. In some embodiments, the method may improve lightening of the skin. In some embodiments, the method may improve brightening effects of the skin. In some embodiments, blackheads in the skin are not stained when applying the topical formulation.

### DETAILED DESCRIPTION

All percentages, parts and ratios used herein are by weight of the total composition and all measurements made are at room temperature, unless otherwise specified. All percentages, parts, and ratios are based upon the total weight of the compositions disclosed herein, unless otherwise specified.

As used herein, the terms “treatment” or “treating” with respect to a skin condition generally mean “having positive effect on a skin condition” and encompass reduction, amelioration, and/or alleviation of at least one symptom of a skin condition, a reduction, amelioration, and/or alleviation in the severity of the skin conditions, or delay, prevention, or inhibition of the progression of the skin condition. Treatment, as used herein, therefore does not require total curing of the condition. A formulation of the present disclosure that is useful for treatment of a skin condition, or a method of treating a skin condition, need only reduce the severity of a skin condition, reduce the severity of symptoms associated therewith, provide improvement to a patient's quality of life, or delay, prevent, or inhibit the onset of one or more symptoms of a skin condition. As used herein, these terms also encompass aesthetic improvements to the skin upon application of the disclosed formulations having a plant-based growth factor.

As used herein, the terms “application,” “apply,” and “applying” with respect to a disclosed topical formulation or method of using a disclosed topical formulation, refer to any manner of administering a topical formulation to the skin, for example, the skin of a person, such as the skin of a patient, which, in medical or cosmetology practice, delivers the formulation to the subject's skin surface. Smearing, rubbing, spreading, spraying a disclosed topical formulation, with or without the aid of suitable devices, on a subject's skin are all included within the scope of the term “application,” as used herein. The term “topical” or “topically” with respect to administration or application of a disclosed skincare formulation refers to epicutaneous administration or application, onto skin. The application can be manually (e.g., directly with the hands) or manipulated with an applicator, cloth, device, roll-on, wipes, unit dose sponge applicators, liquid applied with swabs or cotton balls, impregnated gauze or other substrates, coated silicone sheets or other sheet goods, coated bandages or externally fixed devices, towelettes, individually packages pledgettes or pads, transdermal delivery system, etc. Administration can be self- administration or administration by a medical professional or caregiver.

As used herein, the term “hydroquinone” refers to benzene-1,4-diol or quinol and is a derivative of benzene. “Hydroquinone” refers to the CAS number CAS No. 123-31-9.

As used herein, the phrase “effective amount” refers to an amount of a formulation of the present disclosure, or component thereof, effective to treat a skin condition as noted above, including a range of effects, from a detectable local improvement in an area of topical application to substantial relief of symptoms to an improvement in one or more aesthetic criteria, including, but not limited to, a perceived improvement in apparent skin dryness, age, radiation damage, sun or uv damage, skin tone, weather-beaten appearance, yellowing, redness, dryness, age spots, skin smoothness, brightness, radiance, as well as skin pores becoming less noticeable. The effective amount will vary with the particular condition or conditions being treated, the severity of the condition, the duration of the treatment, the specific components of the composition being used, and other factors. More specifically, the disclosed compositions and formulations provide a method for therapeutic treatment of skin by providing, in some embodiments, a topical formulation including a tyrosine inhibitor, a skin brightener and a resorcinol compound. The topical formulation of the present disclosure does not include hydroquinone. The disclosed compositions, formulations, and methods of use thereof also provide aesthetic improvements in the skin, including but not limited to skin that appears younger, skin exhibiting a more even tone, skin in which the pores are less noticeable, and skin that is judged by the user to be smoother, and/or to be improved with respect to its weather-beaten or aged appearance, yellowing, loss of elasticity, redness, dryness, age spots, and/or skin wrinkles.

According to various embodiments, the present disclosure relates to a topical formulation including a tyrosine inhibitor, a skin brightener, and a resorcinol compound.

In some embodiments of the topical formulation, the tyrosine inhibitor may include a natural tyrosinase inhibitor, a synthetic tyrosinase inhibitor, or a peptide base tyrosinase inhibitor. In some embodiments, the natural tyrosinase inhibitor includes kojic acid, arbutin, ellagic acid, azelaic acid, licorice extract, Vitamin C, ascorbic acid, niacinamide, Vitamin B, mulberry extract, green tea extract, curcumin, aloesin, resveratrol, ferulic acid, glabrene, soybean extract, or a combination thereof. In some embodiments, the synthetic tyrosinase inhibitor may include hydroquinone, monobenzone, monophenol, butylated hydroxyanisole (BHA), butylated hydroxytoluene (BHT), or a combination thereof. In some embodiments, the peptide-based tyrosinase inhibitor may include melanostatine-5, nonapeptide-1 or a combination thereof. In some embodiments, the tyrosine inhibitor may be kojic acid.

In some embodiments, the skin brightener may include a glycosylated derivate of hydroquinone, niacinamide, licorice root extract, or a combination thereof. In some embodiments, the glycosylated derivative of hydroquinone may include arbutin or its derivative. In some embodiments, the arbutin or its derivative may include arbutinase.

In some embodiments, the resorcinol compound may include an alkyl resorcinol, an aromatic resorcinol, or a combination thereof. In some embodiments, the alkyl resorcinol may include at least one of C1-C8 resorcinol. In some embodiments, the alkyl resorcinol may include at least one of C4-C6 resorcinol. In some embodiments, the aromatic resorcinol may include a phenyl alkyl resorcinol. In some embodiments, the phenyl alkyl resorcinol may include at least one of C1-C8 resorcinol.

In some embodiments of the topical formulation, the resorcinol compound may include hexyl resorcinol, butyl resorcinol, phenylethyl resorcinol, or a combination thereof. In some embodiments, the resorcinol compound may include hexyl resorcinol. In some embodiments, the resorcinol compound may include butyl resorcinol. In some embodiments, the resorcinol compound may include phenylethyl resorcinol. In some embodiments, the resorcinol compound may include hexyl resorcinol, butyl resorcinol and phenylethyl resorcinol.

In some embodiments, the topical formulation does not include hydroquinone. In some embodiments, the topical formulation may be substantially free of hydroquinone. As understood herein, “substantially free” refers to less than about 5 wt %, less than about 1 wt %, less than about 0.1 wt %, or less than about 0.01 wt %.

In some embodiments, the topical formulation may include tyrosine inhibitor in an amount of about 0.5 wt % to about 10 wt %, about 1 wt % to about 9 wt %, about 2 wt % to about 8 wt %, about 3 wt % to about 7 wt %, or about 4 wt % to about 6 wt %, based on total weight of the formulation. In some embodiments, the tyrosine inhibitor may be included in an amount of about 0.5 wt %, about 1 wt %, about 2.5 wt %, about 3 wt %, about 4 wt %, about 5 wt %, about 6 wt %, about 7 wt %, about 8 wt %, about 9 wt %. or about 10 wt %, based on total weight of the formulation.

In some embodiments, the topical formulation includes kojic acid in an amount of about 0.5 wt % to about 10 wt %, about 1 wt % to about 9 wt %, about 2 wt % to about 8 wt %, about 3 wt % to about 7 wt %, about 0.5 wt % to about 5 wt %, about 1 wt % to about 5 wt %, about 5 wt % to about 10 wt %, or about 4 wt % to about 6 wt %, based on total weight of the formulation. In some embodiments, the kojic acid may be included in an amount of about 0.5 wt %, about 1 wt %, about 2.5 wt %, about 3 wt %, about 4 wt %, about 5 wt %, about 6 wt %, about 7 wt %, about 8 wt %, about 9 wt %. or about 10 wt %, based on total weight of the formulation.

In some embodiments, the topical formulation may include a skin brightener in an amount of about 1 wt % to about 10 wt %, about 2 wt % to about 9 wt %, about 3 wt % to about 8 wt %, about 4 wt % to about 7 wt %, or about 5 wt % to about 6 wt %, based on total weight of the formulation. In some embodiments, the skin brightener may be included in an amount of about 1 wt %, about 2.5 wt %, about 3 wt %, about 4 wt %, about 5 wt %, about 6 wt %, about 7 wt %, about 8 wt %, about 9 wt %. or about 10 wt %, based on total weight of the formulation.

In some embodiments, the topical formulation may include glycosylated derivative of hydroquinone, such as arbutin, in an amount of about 1 wt % to about 10 wt %, about 2 wt % to about 9 wt %, about 3 wt % to about 8 wt %, about 4 wt % to about 7 wt %, about 1 wt % to about 5 wt %, about 2 wt % to about 10 wt %, about 5 wt % to about 10 wt %, or about 5 wt % to about 6 wt %, based on total weight of the formulation. In some embodiments, the glycosylated derivative of hydroquinone, such as arbutin, may be included in an amount of about 1 wt %, about 2.5 wt %, about 3 wt %, about 4 wt %, about 5 wt %, about 6 wt %, about 7 wt %, about 8 wt %, about 9 wt %. or about 10 wt %, based on total weight of the formulation.

In some embodiments of the topical formulation, the resorcinol compound may include hexyl resorcinol in an amount of about 0.1 wt % to about 10 wt %, about 0.5 wt % to about 9 wt %, about 1 wt % to about 8 wt %, about 2 wt % to about 7 wt %, about 3 wt % to about 6 wt %, or about 4 wt % to about 5 wt %, based on total weight of the formulation. In some embodiments, the hexyl resorcinol may be included in an amount of about 0.1 wt %, about 0.5 wt %, about 1 wt %, about 2.5 wt %, about 4 wt %, about 5 wt %, about 6 wt %, about 7 wt %, about 8 wt %, about 9 wt %, or about 10 wt %, based on total weight of the composition.

In some embodiments of the topical formulation, the resorcinol compound may include butyl resorcinol in an amount of about 0.1 wt % to about 10 wt %, about 0.5 wt % to about 9 wt %, about 1 wt % to about 8 wt %, about 2 wt % to about 7 wt %, about 3 wt % to about 6 wt %, or about 4 wt % to about 5 wt %, based on total weight of the formulation. In some embodiments, the butyl resorcinol may be included in an amount of about 0.1 wt %, about 0.5 wt %, about 1 wt %, about 2.5 wt %, about 4 wt %, about 5 wt %, about 6 wt %, about 7 wt %, about 8 wt %, about 9 wt %, or about 10 wt %, based on total weight of the composition.

In some embodiments of the topical formulation, the resorcinol compound may include phenylethyl resorcinol in an amount of about 0.01 wt % to about 10 wt %, about 0.1 wt % to about 9 wt %, about 1 wt % to about 8 wt %, about 2 wt % to about 7 wt %, about 3 wt % to about 6 wt %, about 0.01 wt % to about 5 wt %, about 1 wt % to about 10 wt %, about 2 wt % to about 10 wt %, or about 4 wt % to about 5 wt %, based on total weight of the formulation. In some embodiments, the phenylethyl resorcinol may be included in an amount of about 0.01 wt %, about 0.1 wt %, about 0.5 wt %, about 1 wt %, about 2.5 wt %, about 4 wt %, about 5 wt %, about 6 wt %, about 7 wt %, about 8 wt %, about 9 wt %, or about 10 wt %, based on total weight of the composition.

In some embodiments of the topical formulation, the resorcinol compound may include hexyl resorcinol in an amount of about 0.1 wt % to about 10 wt %, about 0.5 wt % to about 9 wt %, about 1 wt % to about 8 wt %, about 2 wt % to about 7 wt %, about 3 wt % to about 6 wt %, or about 4 wt % to about 5 wt %, based on total weight of the formulation; butyl resorcinol in an amount of about 0.1 wt % to about 10 wt %, about 0.5 wt % to about 9 wt %, about 1 wt % to about 8 wt %, about 2 wt % to about 7 wt %, about 3 wt % to about 6 wt %, or about 4 wt % to about 5 wt %, based on total weight of the formulation; and phenylethyl resorcinol in an amount of about 0.01 wt % to about 10 wt %, about 0.1 wt % to about 9 wt %, about 1 wt % to about 8 wt %, about 2 wt % to about 7 wt %, about 3 wt % to about 6 wt %, or about 4 wt % to about 5 wt %, based on total weight of the formulation.

In some embodiments, the topical formulation may include an ascorbate component. In some embodiments, the ascorbate component may include ascorbic acid, derivatives thereof, or combinations thereof. In some embodiments, the ascorbate component comprises ascorbic acid, ascorbyl palmitate, sodium ascorbate, potassium ascorbate, ammonium ascorbate, triethanolamine ascorbate, ascorbyl phosphate or magnesium ascorbyl phosphate, ascorbic acid polypeptides, ascorbyl glucosamine, ascorbic acid polymers, esters of ascorbic acid, amides of ascorbic acid, L-ascorbic acid, tetrahexyldecyl ascorbate or combinations thereof. In some embodiments, the ascorbate component may include ascorbic acid.

In some embodiments, the topical formulation may include an ascorbate component, such as ascorbic acid, in an amount of about 1 wt % to about 30 wt %, about 1 wt % to about 20 wt %, about 1 wt % to about 10 wt %, about 5 wt % to about 30 wt %, about 10 wt % to about 30 wt %, about 20 wt % to about 30 wt %, about 5 wt % to about 25 wt %, or about 10 wt % to about 20 wt %, based on total weight of the formulation.

In some embodiments, the topical formulation may include a solvent. In some embodiments, the solvent may include water, ethoxydiglycol, alcohol, alkanediol, glycerin, an aromatic alcohol, ethoxylated or propoxylated diglycol, PPG-2 methyl ether, PPG-3 propyl ether, propylene glycol butyl ether, PPG-2 butyl ether, phenoxyisopropanol, butoxyethanol, butoxydiglycol, methoxdiglycol, phenoxyethanol, PPG-3 butyl ether, PPG-2 propyl ether, propylene glycol propyl ether, or dipropylene glycol dimethyl ether, or a combination thereof. In some embodiments, the solvent may include water, ethoxydiglycol, alcohol, alkanediol, glycerin, an aromatic alcohol, or a combination thereof.

In some embodiments, the topical formulation may include water in an amount of about 20 wt % to about 80 wt %, about 25 wt % to about 75 wt %, about 30 wt % to about 70 wt %, about 35 wt % to about 65 wt %, about 40 wt % to about 60 wt %, or about 45 wt % toa bout 55 wt %, based on total weight of the formulation.

In some embodiments, the topical formulation may include ethoxydiglycol in an amount of about 1 wt % to about 25 wt %, about 3 wt % to about 22 wt %, about 5 wt % to about 20 wt %, about 7 wt % to about 18 wt %, or about 10 wt % to about 15 wt %, based on total weight of the formulation.

In some embodiments, the alcohol may include ethanol. In some embodiments, the topical formulation may include alcohol in an amount of about 1 wt % to about 50 wt %, about 5 wt % about to about 45 wt %, about 10 wt % to about 40 wt %, about 15 wt % to about 35 wt %, about 20 wt % to about 30 wt %, based on total weight of the formulation.

In some embodiments, the alkanediol may include ethylene glycol, propylene glycol, butylene glycol, or a combination thereof. In some embodiments, the alkanediol may include butylene glycol. In some embodiments, the alkanediol, such as butylene glycol, may be included in the topical formulation in an amount of about 1 wt % to about 30 wt %, about 5 wt % to about 27 wt %, about 8 wt % to about 25 wt %, about 10 wt % to about 20 wt %, about 1 wt % to about 15 wt %, about 5 wt % to about 20 wt %, about 15 wt % to about 30 wt %, or about 12 wt % to about 18 wt %, based on total weight of the formulation.

In some embodiments, the topical formulation may include glycerin. In some embodiments, the glycerin may be included in an amount of about 0.001 wt % to about 20 wt %, about 0.005 wt % to about 19 wt %, about 0.01 wt % to about 18 wt %, about 0.1 wt % to about 17 wt %, about 1 wt % to about 15 wt %, about 2 wt % to about 13 wt %, about 3 wt % to about 12 wt %, about 0.001 wt % to about 15 wt %, about 0.001wt % to about 10 wt %, about 10 wt % to about 20 wt %, about 5 wt % to about 20 wt %, or about 5 wt % to about 10 wt %, based on total weight of the formulation.

In some embodiments, the topical formulation may include aromatic alcohol. In some embodiments, the aromatic alcohol may include benzyl alcohol. In some embodiments, the topical formulation may include the aromatic alcohol, i.e. benzyl alcohol, in an amount of about 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, about 1 wt % to about 4 wt %, about 0.001 wt % to about 5 wt %, about 0.01 wt % to about 5 wt %, about 5 wt % to about 10 wt %, or about 2 wt % to about 3 wt %, based on total weight of the formulation.

In some embodiments, the topical formulation may further include a keratolytic. In some embodiments, the keratolytic may include salicylic acid, urea, lactic acid, glycolic acid or a combination thereof. In some embodiments, the keratolytic may include salicylic acid.

In some embodiments, the topical formulation may include the keratolytic acid, such as salicylic acid, in an amount of about 0.1 wt % to about 10 wt %, about 0.5 wt % to about 9 wt %, about 1 wt % to about 8 wt %, about 2 wt % to about 7 wt %, about 3 wt % to about 6 wt %, about 0.1wt % to about 5 wt %, about 1 wt % to about 5 wt %, about 5 wt % to about 10 wt %, or about 4 wt % to about 5 wt %, based on total weight of the topical formulation.

In some embodiments, the topical formulation may further include an emulsifier. In some embodiments, the emulsifier may include polysorbate 20, polysorbate 40, polysorbate 60, potassium cetyl sulfate, lauryl alcohol, and polyethylene glycol, or a combination thereof. In some embodiments, the emulsifier may include lauryl alcohol and polyethylene glycol.

In some embodiments, the topical formulation may include an emulsifier, such as the lauryl alcohol and polyethylene glycol, in an amount of about 0.01 wt % to about 10 wt %, about 0.05 wt % to about 9 wt %, about 0.1 wt % to about 8 wt %, about 0.5 wt % to about 7 wt %, about 1 wt % to about 6 wt %, about 2 wt % to about 5 wt %, or about 3 wt % to about 4 wt %, based on total weight of the formulation.

In some embodiments, the topical formulation may further include potassium hydroxide. In some embodiments, the potassium hydroxide may be included in an amount of about 0.001 wt % to about 5 wt %, about 0.005 wt % about 4.5 wt %, about 0.01 wt % to about 4 wt %, about 0.05 wt % to about 3 wt %, about 0.1 wt % to about 2.5 wt %, 0.001 wt % to about 2.5 wt %, about 2.5 wt % to about 5 wt %, or about 0.5 wt % to about 2 wt %, based on total weight of the formulation.

In some embodiments, the topical formulation may further include a chelating agent. In some embodiments, the chelating agent may include disodium ethylenediaminetetraacetic acid (EDTA), diethylenetriaminepentaacetic acid (DTPA), N-(hydroxyethyl)-ethylenediaminetriacetic acid (HEDTA), nitrilotriacetic acid (NTA), or a combination thereof. In some embodiments, the chelating agent may include disodium EDTA.

In some embodiments, the topical formulation may include the chelating agent, such as disodium EDTA, in an amount of about 0.0001 wt % to about 0.5 wt %, about 0.0005 wt % to about 0.4 wt %, about 0.001 wt % to about 0.3 wt %, about 0.01 wt % to about 0.25 wt %, or about 0.1 wt % to about 0.2 wt %, based on total weight of the formulation.

In some embodiments, the topical formulation may further include an antioxidant. In some embodiments, the antioxidant may include a hydroxy acid. In some embodiments, the hydroxy acid may include citric acid, lactic acid, malic acid, or a combination thereof. In some embodiments, the hydroxy acid may include citric acid. In some embodiments, the antioxidant, such as citric acid, may be included in an amount of about 0.001 wt % to about 1 wt %, about 0.005 wt % to about 0.9 wt %, about 0.01 wt % to about 0.8 wt %, about 0.05 wt % to about 0.7 wt %, about 0.1 wt % to about 0.6 wt %, about 0.001 wt % to about 0.5 wt %, about 0.5 wt % to about 1 wt %, or about 0.2 wt % to about 0.5 wt %, based on total weight of the formulation.

In some embodiments, the topical formulation may further include a botanical extract. In some embodiments, the botanical extract may include a thyme herb, mulberry extract, willow bark extract, elderberry, Echinacea, cucumber base extract, or a combination thereof. In some embodiments, the botanical extract may be included in an amount of about 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, about 1 wt % to about 4 wt %, or about 2 wt % to about 3 wt %, based on total weight of the formulation.

In some embodiments, the topical formulation may further include a bark extract. In some embodiments, the bark extract may be willow bark extract. In some embodiments, the bark extract may be included in an amount of about 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, or about 1 wt % to about 4 wt % based on total weight of the formulation.

In some embodiments, the topical formulation may further include a root extract. In some embodiments, the root extract may be rumex crispus root extract, licorice root extract, or a combination thereof. In some embodiments, the root extract (individually or collectively) may be included in an amount of about 0.0001 to about 20%, about 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, or about 1 wt % to about 4 wt % based on total weight of the formulation.

In some embodiments, the topical formulation may further include a flower extract. In some embodiments, the flower extract may be matricaria recutita flower extract. In some embodiments, the flower extract may be included in an amount of about 0.0001 wt % to about 10 wt %, about 0.0005 wt % to about 9 wt %, about 0.001 wt % to about 8 wt %, about 0.05 wt % to about 7 wt %, about 0.1 wt % to about 6 wt %, about 0.5 wt % to about 5 wt %, or about 1 wt % to about 4 wt % based on total weight of the formulation.

In some embodiments, the topical formulation may further include a leaf extract. In some embodiments, the leaf extract may be Arctostaphylos uva-ursi leaf extract. In some embodiments, the leaf extract may be included in an amount of about 0.0001 wt % to about 10 wt %, about 0.0005 wt % to about 9 wt %, about 0.001 wt % to about 8 wt %, about 0.05 wt % to about 7 wt %, about 0.1 wt % to about 6 wt %, about 0.5 wt % to about 5 wt %, or about 1 wt % to about 4 wt % based on total weight of the formulation.

In some embodiments, the topical formulation may further include a polyphenol. In some embodiments, the polyphenol may be a green tea extract. In some embodiments, the green tea extract may include Camellia sinensis extract. In some embodiments, the polyphenol, such as Camellia sinensis extract may be included in an amount of about 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, or about 1 wt % to about 4 wt % based on total weight of the formulation.

In some embodiments, the topical formulation may further include a cosmetically acceptable excipient. Exemplary cosmetically acceptable excipients, include without limitations, epidermal penetration enhancer, solvent, mild surfactants, oil bodies, emulsifiers, pearlescent waxes, consistency regulators, thickeners, rheology modifiers, suspending agents, preservatives, super fatting agents, stabilizers, polymers, silicone or siloxane compounds, fats, waxes, lecithins, phospholipids, additional antioxidants, swelling agents, solubilizers, perfume oils, dyes, fatty alcohols, esters of fatty acids, adjuvants, bacteria-inhibiting agents, colorants, natural gums, a booster, a hydrator, an anti-inflammatory agent, a skin soother, an enzyme blocker, or combinations thereof.

In some embodiments, the cosmetically acceptable excipient may include Laureth-4. In some embodiments, Laureth-4 may be included in the topical formulation in an amount of about 0.001 wt % to 10 wt %, about 0.001 wt % to about 5 wt %, about 5 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, about 1 wt % to about 4 wt %, or about 2 wt % to about 3 wt % based on total weight of the formulation.

In some embodiments, the cosmetically acceptable excipient may include a natural gum. The natural gum may include guar gum, carob gum, konjac gum, xanthan gum, sclerotium gum, acacia gum, cellulose gum, or a combination thereof.

In some embodiments, the cosmetically acceptable excipient may include a booster. Suitable boosters may include niacinamide, cysteamine, tranexamic acid, azelaic acid, a retinoid, terminalia chebula fruit extract, a keroltyic, a peptide, reseveratrol, green tea extract, caffeine, or a combination thereof.

In some embodiments, the cosmetically acceptable excipient may include a hydrator. Suitable hydrators may include hyaluronic acid, glycerin, aloe vera, panthenol, sodium pyrrolidone carboxylic acid (pca), propylene glycol, butylene glycol, sorbitol, beta-glucan, allantoin, or a combination thereof.

In some embodiments, the cosmetically acceptable excipient may include an anti-inflammatory agent. Suitable anti-inflammatory agents may include aloe vera, green tea extract, or a combination thereof.

In some embodiments, the cosmetically acceptable excipient may include a skin soother. Suitable skin soothers may include allantoin, a chamomile extract, or a combination thereof.

In some embodiments, the cosmetically acceptable excipient may include a further emulsifier from described above. The further emulsifier may include PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate, or a combination thereof.

Additional emulsifiers may include phosphate esters and the salts thereof such as cetyl phosphate (Amphisol® A), diethanolamine cetyl phosphate (Amphisol® DEA), potassium cetyl phosphate (Amphisol® K), sodium cetearyl sulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobutene. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

In some embodiments, the cosmetically acceptable excipient may include an additional antioxidant. The additional antioxidant may include Vitamin E. Suitable forms of Vitamin E that may be included are alpha, beta, delta, and gamma tocopherols, and alpha, beta, delta, and gamma tocotrienols, or combinations thereof.

In some embodiments, the cosmetically acceptable excipient may include a preservative. Suitable preservatives may include phenoxyethanol, a solution of paraben, pentanediol and sorbic acid, as well as silver complexes which are known under the commercial reference Surfacine® and other classes of substances set out in annex 6, parts A and B of the cosmetic regulations, i.e. a suitable preservative.

In some embodiments, the cosmetically acceptable excipient may include a natural oil. Suitable natural oils may include jojoba oil, a tea tree oil, or a combination thereof.

In some embodiments, the cosmetically acceptable excipient may include an enzyme blocker. The enzyme blocker may include tretinoin.

In some embodiments, the cosmetically acceptable excipient may include a perfume oil. In some embodiments, the perfume oil may include mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include mainly the terpenes and balsams.

Essential oils of relatively low volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

In certain embodiments, the cosmetically acceptable excipient may include a perfume oil that is an essential oil which may be selected from lavender oil, a bergamot oil, a eucalyptus oil, a chamomile oil, a melaleuca oil, or a combination thereof.

In some embodiments, the cosmetically acceptable excipient may be a fatty alcohol. The fatty alcohol may be a Guerbet alcohol based on fatty alcohols having from 6 to 18, preferably from 8 to 10 carbon atoms including cetyl alcohols, stearyl alcohol, cetearyl alcohol, oleyl alcohol, octyldodecanol, benzoate of C12-C15 alcohols, acetylated lanolin alcohol, etc.

In some embodiments, the cosmetically acceptable excipient may be an ester of fatty acids. Esters of linear C₆-C₂₄ fatty acids with linear C₃-C₂₄ alcohols, esters of branched C₆-C₁₃carboxyl acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol and brassidyl alcohol and technical-grade mixtures thereof (obtained, for example, in the high-pressure hydrogenation of technical-grade methyl esters based on fats and oils or aldehydes from Roelen's oxosynthesis and as monomer fractions in the dimerization of unsaturated fatty alcohols).

Examples of such ester oils are isopropyl myristate, isopropyl palmitate, isopropyl stearate, isopropyl isostearate, isopropyl oleate, n-butyl stearate, n-hexyl laurate, n-decyl oleate, isooctyl stearate, iso-nonylstearate, isononyl isononanoate, 2-ethylhexylpalmitate, 2-hexyllaurate, 2-hexyldecyl stearate, 2-octyldodecylpalmitate, oleyloleate, oleylerucate, erucyloleate, erucylerucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, octyl hydroxystearate, etc.

In some embodiments, the cosmetically acceptable excipient may include other adjuvants. The other adjuvants may include diethylhexyl 2,6-naphthalate, di-n-butyl adipate, di(2-ethylhexyl)-adipate, di(2-ethyl hexyl)-succinate and diisotridecyl acelaat, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate. Esters of C₆-C₂₄ fatty alcohols and/or Guerbet alcohols with aromatic carboxylic acids, saturated and/or unsaturated, especially benzoic acid, esters of C₂-C₁₂ dicarboxylic acids with linear or branched alcohols having from 1 to 22 carbon atoms or polyols having from 2 to 10 carbon atoms and from 2 to 6 hydroxy groups.

In some embodiments, the cosmetically acceptable excipient may include natural or synthetic triglycerides including glyceryl esters and derivatives. The natural or synthetic triglycerides may include di-or triglycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borage oil, etc.

In some embodiments, the cosmetically acceptable excipient may include waxes. The waxes may include esters of long-chain acids and alcohols as well as compounds having wax-like properties, e.g., carnauba wax, beeswax (white or yellow), lanolin wax, candelilla wax, ozokerite, japan wax, paraffin wax, microcrystalline wax, ceresin, cetearyl esters wax, synthetic beeswax, etc. Also, hydrophilic waxes as Cetearyl Alcohol or partial glycerides. The waxes may also include pearlescent waxes. The pearlescent waxes may include alkylene glycol esters, especially ethylene glycol distearate; fatty acid alkanolamides, especially coco fatty acid diethanolamide; partial glycerides, especially stearic acid monoglyceride; esters of polyvalent, unsubstituted or hydroxy- substituted carboxylic acids with fatty alcohols having from 6 to 22 carbon atoms, especially long- chained esters of tartaric acid; fatty substances, for example fatty alcohols, fatty ketones, fatty aldehydes, fatty ethers and fatty carbonates, which in total have at least 24 carbon atoms, especially lauryl and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures thereof.

In some embodiments, the cosmetically acceptable excipient may include hydrocarbon oils. The hydrocarbon oil may include mineral oil (light or heavy), petrolatum (yellow or white), microcrystalline wax, paraffinic and isoparaffinic compounds, hydrogenated isoparaffinic molecules as polydecenes and polybutene, hydrogenated polyisobutene, squalane, isohexadecane, isododecane and others from plant and animal kingdom.

In some embodiments, the cosmetically acceptable excipient may include silicones or siloxanes. The silicones or siloxanes may include dimethylpolysiloxanes, methylphenylpolysiloxanes, cyclic silicones, and also amino- , fatty acid- , alcohol- , polyether- , epoxy- , fluorine- , glycoside- and/or alkyl-modified silicone compounds, which at room temperature may be in either liquid or resinous form. Linear polysiloxanes, dimethicone (Dow Corning 200 fluid, Rhodia Mirasil DM), dimethiconol, cyclic silicone fluids, cyclopentasiloxanes volatiles (Dow Corning 345 fluid), phenyltrimethicone (Dow Corning 556 fluid). Also suitable are simethicones, which are mixtures of dimethicones having an average chain length of from 200 to 300 dimethylsiloxane units with hydrogenated silicates

In some embodiments, the cosmetically acceptable excipient may include an additional emulsifier. The emulsifier may include carboxylic acids and their salts: alkaline soap of sodium, potassium and ammonium, metallic soap of calcium or magnesium, organic basis soap such as Lauric, palmitic, stearic and oleic acid etc. Alkyl phosphates or phosphoric acid esters, acid phosphate, diethanolamine phosphate, potassium cetyl phosphate. Ethoxylated carboxylic acids or polyethylene glycol esters, PEG-n acylates. Linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group. Fatty alcohol polyglycol ether such as laureth-n, ceteareth-n, steareth-n, oleth-n. Fatty acid polyglycolether such as PEG-n stearate, PEG-n oleate, PEG-n cocoate. Monoglycerides and polyol esters. C12-C22 fatty acid mono- and di-esters of addition products of from 1 to 30 mol of ethylene oxide with polyols. Fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters, glycerol and saccharose esters such as sucro glycerides. Sorbitol and sorbitan, sorbitan mono-and di-esters of saturated and unsaturated fatty acids having from 6 to 22 carbon atoms and ethylene oxide addition products. Polysorbate-n series, sorbitan esters such as sesquiisostearate, sorbitan, PEG-(6)-isostearate sorbitan, PEG-(10)-sorbitan laurate, PEG-17-dioleate sorbitan. Glucose derivatives, C8-C22 alkyl-mono and oligo-glycosides and ethoxylated analogues with glucose being preferred as the sugar component. O/W emulsifiers such as methyl gluceth-20 sesquistearate, sorbitan stearate/sucrose cocoate, methyl glucose sesquistearate, cetearyl alcohol/cetearyl glucoside. W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated paraffins, sulfonated tetrapropyene sulfonate, sodium lauryl sulfates, ammonium and ethanolamine lauryl sulfates, lauryl ether sulfates, sodium laureth sulfates, sulfosuccinates, acetyl isothionates, alkanolamide sulfates, taurines, methyl taurines, imidazole sulfates. Polysiloxane/polyalkyl/polyether copolymers and derivatives, dimethicone, copolyols, silicone polyethylene oxide copolymer, silicone glycol copolymer. Propoxylated or POE-n ethers (Meroxapols), Polaxamers or poly(oxyethylene)m-block-poly(oxypropyl ene)n-block(oxyethylene). Zwitterionic surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkyl betaine, N-alkylaminobetaines. Alkylimidazolines, alkylopeptides, lipoaminoacides, and self-emulsifying bases.

Nonionic bases such as PEG-6 beeswax (and) PEG-6 stearate (and) polyglyceryl-2-isostearate, glyceryl stearate (and) PEG-100 stearate, PEG-5 glyceryl stearate, sorbitan oleate (and) polyglyceryl-3 ricinoleate, sorbitan stearate and sucrose cocoate, glyceryl stearate and laureth-23, cetearyl alcohol and ceteth-20, cetearyl alcohol and polysorbate 60 and PEG-150 and stearate-20, cetearyl alcohol and cetearyl polyglucoside, cetearyl alcohol and ceteareth-20, cetearyl alcohol and PEG-40 castor oil, cetearyl alcohol and PEG-40 castor oil and sodium cetearyl sulfate, stearyl alcohol and steareth-7 and steareth-10, cetearyl alcohol and szeareth-7 and steareth-10, glyceryl stearate and PEG-75 stearate, propylene glycol ceteth-3 acetate, propylene glycol isoceth-3 acetate, cetearyl alcohol and ceteth-12 and oleth-12, PEG-6 stearate and PEG-32 stearate, PEG-6 stearate and ceteth-20 and steareth-20, PEG-6 stearate and ceteth-20 and glyceryl stearate and steareth-20, glyceryl stearate and ceteareth-20.

Anionic alkaline bases such as PEG-2 stearate SE, glyceryl stearate SE, propylene glycol stearate. Anionic acid bases such as cetearyl Alcohol and Sodium cetearyl sulfate, cetearyl alcohol and sodium lauryl sulfate, trilaneth-4 phosphate and glycol stearate and PEG-2 stearate, glyceryl stearate and sodium lauryl Sulfate. Cationic acid bases such as cetearyl alcohol and cetrimonium bromide.

In some embodiments, the cosmetically acceptable excipients may include super-fatting agents. The super-fatting agents may include lanolin and lecithin and also polyethoxylated or acetylated lanolin and lecithin derivatives, polyol fatty acid esters, monoglycerides and fatty acid alkanolamides, the latter simultaneously acting as foam stabilizers.

In some embodiments, the cosmetically acceptable excipient may include a mild surfactant. The mild surfactant may include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, .alpha.-olefin sulfonates, ethercarboxylic acids, alkyl oligoglucosides, fatty acid glucamides, alkylamidobetaines and/or protein fatty acid condensation products, the latter preferably being based on wheat proteins.

In some embodiments, the cosmetically acceptable excipient may include consistency regulators/thickeners or rheology modifiers. The consistency regulators/thickeners or rheology modifiers may include silicium dioxide, magnesium silicates, aluminium silicates, polysaccharides or derivatives thereof for example hyaluronic acid, xanthan gum, guar-guar, agar-agar, alginates, carrageenan, gellan, pectines, or modified cellulose such as hydroxycellulose, hydroxypropylmethylcellulose. In addition polyacrylates or homopolymer of reticulated acrylic acids and polyacrylamides, carbomer (CARBOPOL types 980, 981, 1382, ETD 2001, ETD2020, ULTREZ 10) or SALCARE range such as SALCARE SC80 (steareth-10 allyl ether/acrylates copolymer), Salcare SC81 (acrylates copolymer), Salcare SC91 and Salcare AST (sodium acrylates copolymer/PPG-1 trideceth-6), SEPIGEL 305 (polyacrylamide/laureth-7), SIMULGEL NS and SIMULGEL EG (hydroxyethyl acrylate/sodium acryloyldimethyl taurate copolymer), STABILEN 30 (acrylates/vinyl isodecanoate crosspolymer), PEMULEN TR-1 (acrylates/C10-30 alkyl acrylate crosspolymer), LUVIGEL EM (sodium acrylates copolymer), ACULYN 28 (acrylates/beheneth-25 methacrylate copolymer), etc.

In some embodiments, the cosmetically acceptable excipient may include a polymer. The polymer may be anionic, zwitterionic, amphoteric and non-ionic polymers, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyl-trimethylammonium chloride/acrylate copolymers, octyl acrylamide/methyl methacrylate-tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinyl caprolactam terpolymers and also optionally derivatized cellulose ethers and silicones.

In some embodiments, the cosmetically acceptable excipient may include a bacteria- inhibiting agent. The bacteria-inhibiting agent may also be a preservative that has a specific action against gram-positive bacteria, such as 2,4,4′-trichloro-2′-hydroxydiphenyl ether, chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorizing agent of interest is the terpene alcohol farnesol (3,7,11-trimethyl-2,6,10-dodecatrien-1-ol), which is present in lime blossom oil. Glycerol monolaurate has also proved to be a bacteriostatic agent.

In some embodiments, the topical formulation may be a moisturizer, an elixir, a serum, a face cream, a gel, a toner, a face mask, a cleanser or a sunscreen.

In another embodiment, a delivery system is provided. The delivery system may include an applicator pad infused with the topical formulation described herein.

In yet another embodiment, a method of treating skin is provided. The method includes applying a topical formulation as described herein. The method may include applying with an applicator pad or an applicator device. The applicator device may include a roll-on device. The applicator pad may include a cotton pad, a microfiber pad, or a foam pad. In some embodiments, the method may improve lightening of the skin. In some embodiments, the method may improve brightening effects of the skin. In some embodiments, the blackheads in the skin may not be stained when applying the topical formulation.

In some embodiments, the method of treating includes treating, without limitation, the following conditions: age spots, hyperpigmentation, skin surface irregularities, roasacea, acne, psoriasis, yellowing, redness, and other damaging skin conditions. In some embodiments, the method is directed to brightening a skin of a subject by administering to the skin a topical formulation according to the present disclosure, wherein the skin is brightened after administration of the topical formulation.

In some embodiments, the topical formulation may improve the effect of skin brightness and/or lightening; decrease the appearance of post inflammatory hyperpigmentation; improve the appearance of skin tone and/or dark spots; improve signs of aging; reduce the appearance of melasma; stimulate collagen; reduce appearance of acne and blackheads; decrease appearance of pores and texture; improve overall tolerance level based on clinical grading; and/or improve confidence score in overall skin assessed by subject.

In some embodiments, the topical formulation of the present disclosure had statistically significant (p<0.0001) differences between baseline (i.e., time=0 weeks) and 12 weeks of use. In some embodiments, the topical formulation may have statistically significant differences to improve brightness/radiance, reduce pores/texture, improve skin tone and/or reduce dark spots.

As used herein, the terms “treating” or “treatment with respect to a skin condition generally mean “having positive effect on a skin condition” and encompass reduction, amelioration, and/or alleviation of at least one symptom of a skin condition, a reduction, amelioration, and/or alleviation in the severity of the skin conditions, or delay, prevention, or inhibition of the progression of the skin condition, or the perception thereof. Treatment, as used herein, therefore does not require total curing of the condition. A formulation of the present disclosure that is useful for treatment of a skin condition, or a method of treating a skin condition, need only reduce the severity of a skin condition, reduce the severity of symptoms associated therewith, provide improvement to a patient's quality of life, or delay, prevent, or inhibit the onset of one or more symptoms of a skin condition. As used herein, these terms also encompass aesthetic improvements to the skin upon application of the disclosed formulations having a combination of, for example, tyrosine inhibitor, skin brightening agent, and enzyme.

As used herein, the phrase “effective amount” refers to an amount of a topical composition of the present disclosure, or component thereof, effective to treat a skin condition as noted above, including a range of effects, from a detectable local improvement in an area of topical application to substantial relief of symptoms to an improvement in one or more aesthetic criteria, including, but not limited to, a perceived improvement in damage from free radicals from sunlight (UVB, UVA, Visible Light), HEV (blue) light, Infrared (IR), pollution, irritants, allergens, and various environmental toxins, skin wrinkles, appearance of fine lines, skin roughness, skin sagging, skin firmness, skin elasticity, age spots, hyperpigmentation, scars, skin surface irregularities, rosacea, acne, psoriasis, skin's regenerative and renewal process, weather-beaten appearance, yellowing, redness, dryness, ichthyosis, and other damaging skin conditions.

The effective amount will vary with the particular condition or conditions being treated, the severity of the condition, the duration of the treatment, the specific components of the composition being used, and other factors. In certain embodiments, the topical compositions described herein are suitable for administration by frequent periodic application, such as by a once, twice, thrice or four times daily application or more, e.g., for a duration of at least 1 day, at least 3 days, at least 5 days, at least 7 days, at least 10 days, at least 14 days, at least 21 days, at least 30 days, and so on. Accordingly, in certain embodiments, the methods described herein further include periodically repeating the administration of the topical composition.

### ILLUSTRATIVE EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1—Formulation Including Hexylresorcinol

A formulation in accordance with embodiments described herein was formulated into an application having the composition described in Table 1 below. The formulation of Example 1 may be applied to the skin.

TABLE 1
Formulation 1 including hexylresorcinol

| Ingredient | Amount (wt. %) |
|---|---|
| Purified Water | 20-80 |
| Ethoxydiglycol | 1-25 |
| Alcohol Denatured | 1-50 |
| Ascorbic Acid | 1-30 |
| Arbutin | 1-10 |
| Kojic Acid | 0.5-10 |
| Butylene Glycol | 1-30 |
| Salicylic Acid | 0.1-10 |
| Glycerin | 0.001-20 |
| Laureth-4 | 0.001-10 |
| Hexylresorcinol | 0.1-10 |
| Potassium hydroxide | 0.001-5 |
| Benzyl Alcohol | 0.001-10 |
| Disodium EDTA | 0.001-0.5 |
| Rumex Crispus Root Extract | 0.0001-1 |
| Salix Alba (Willow) Bark Extract | 0.001-10 |
| Camellia Sinensis Leaf Extract | 0.0001-10 |
| Arctostaphylos Uva-Ursi Leaf Extract | 0.0001-10 |
| Matricaria Recuttia Flower Extract | 0.0001-10 |
| Glycyrrhiza Glabra (Licorice) Root Extract | 0.0001-10 |

### Example 2—Formulation Including Butylresorcinol

A formulation in accordance with embodiments described herein was formulated into an application pad having the composition described in Table 2 below.

TABLE 2
Formulation 2 including butylresorcinol

| Ingredient | Amount (wt. %) |
|---|---|
| Purified Water | 20-80 |
| Ethoxydiglycol | 1-25 |
| Alcohol Denatured | 1-50 |
| Ascorbic Acid | 1-30 |
| Arbutin | 1-10 |
| Kojic Acid | 0.5-10 |
| Butylene Glycol | 1-30 |
| Salicylic Acid | 0.1-10 |
| Glycerin | 0.001-20 |
| Laureth-4 | 0.0001-10 |
| Potassium hydroxide | 0.0001-5 |
| 4-Butylresorcinol | 0.1-10 |
| Benzyl Alcohol | 0.001-10 |
| Disodium EDTA | 0.0001-0.5 |
| Citric Acid | 0.0001-1 |
| Rumex Crispus Root Extract | 0.0001-1 |
| Salix Alba (Willow) Bark Extract | 0.001-10 |
| Camellia Sinensis leaf extract | 0.001-10 |
| Acrtostaphylos Uva-Ursi leaf extract | 0.0001-10 |
| Matricaria Recutita Flower Extract | 0.0001-10 |
| Glycyrrhiza Glabra (Licorice) Root Extract | 0.0001-10 |

### Example 3—Formulation with Phenylethyl Resorcinol

A formulation in accordance with embodiments described herein was formulated into an application pad having the composition described in Table 3 below.

TABLE 3
Formulation 3 with phenylethyl resorcinol

| Ingredient | Amount (wt. %) |
|---|---|
| Purified Water | 20-80 |
| Ethoxydiglycol | 1-25 |
| Alcohol Denatured | 1-50 |
| Ascorbic Acid | 1-30 |
| Arbutin | 1-10 |
| Kojic Acid | 0.5-10 |
| Butylene Glycol | 1-30 |
| Salicylic Acid | 0.1-10 |
| Glycerin | 0.001-20 |
| Laureth-4 | 0.001-10 |
| Potassium hydroxide | 0.001-5 |
| Phenylethyl resorcinol | 0.01-10 |
| Benzyl alcohol | 0.001-10 |
| Disodium EDTA | 0.001-0.5 |
| Citric Acid | 0.001-1 |
| Rumex Crispus Root Extract | 0.0001-1 |
| Salix Alba (Willow) bark extract | 0.001-10 |
| Camellia Sinensis leaf extract | 0.001-10 |
| Arctostaphylos Uva-Ursi leaf extract | 0.0001-10 |
| Matricaria Recutita Flower extract | 0.0001-10 |
| Glycyrrhiza Glabra (Licorice) Root extract | 0.0001-10 |

### Example 4—Formulation with Combination of 3 Resorcinol Compounds

A formulation in accordance with embodiments described herein was formulated into an application having the composition described in Table 4 below.

TABLE 4
Formulation 4 with Hexyl-, butyl- and phenylethyl resorcinol

| Ingredient | Amount (wt. %) |
|---|---|
| Purified Water | 20-80 |
| Ethoxydiglycol | 1-25 |
| Alcohol Denatured | 1-50 |
| Ascorbic Acid | 1-30 |
| Arbutin | 1-10 |
| Kojic Acid | 0.5-10 |
| Butylene Glycol | 1-30 |
| Salicylic Acid | 0.1-10 |
| Glycerin | 0.001-20 |
| Laureth-4 | 0.001-10 |
| Hexylresorcinol | 0.1-10 |
| Phenylethyl resorcinol | 0.001-10 |
| 4-Butylresorcinol | 0.1-10 |
| Potassium hydroxide | 0.01-5 |
| Benzyl alcohol | 0.001-10 |
| Disodium EDTA | 0.001-0.5 |
| Citric Acid | 0.001-1 |
| Rumex Crispus Root Extract | 0.001-1 |
| Salix Alba (Willow) bark extract | 0.001-10 |
| Camellia Sinensis leaf extract | 0.0001-10 |
| Arctostaphylos Uva-Ursi leaf extract | 0.0001-10 |
| Matricaria Recutita Flower Extract | 0.0001-10 |
| Glycyrrhiza Glabra (Licorice) Root Extract | 0.0001-10 |

### Consumer Test Results

A study was performed to assess the efficacy and sensitivity of the topical formulations according to the present disclosure that does not include hydroquinone. The topical formulation of the present study includes a combination of hexylresorcinol, butylresorcinol, phenylethyl resorcinol into a soluble form and combining it with kojic acid and arbutin to treat pigmentation caused by sun damage, melasma, acne, sun spots or age spots. The study included Formulations 1-4 as described above to investigate the efficacy and tolerability of the formulations for improving the appearance of pigmentation. The formula tested in the presented in the study ensured compliance with the CARES Act by not including hydroquinone.

The formulations of the present study was also tested in a delivery system in pad form to penetrate deep into the skin at the same time which causes early and better results than previous formulas.

A total of 20 medical experts between the ages of 25-50 (2 male and 18 females) were enrolled in the study. Subjects were treated with the applicator pad including a formulation according to the present disclosure. Subjects were asked to apply the pads nightly 5 times per week with 2 off days per week to focus on hydration. The subjects were instructed to continue with their normal skincare routine but no other products were to be used on the face.

### Objectives

The objectives of the study were to determine if the use of a blend of resorcinols combined with koji acid and arbutin.
Improves the effects of skin brightness and lightening through image analysis weekly
Decreases the appearance of pigmentation through image analysis weekly
Improves the appearance of post inflammatory hyperpigmentation (PIH) through image analysis weekly
Improves the appearance of skin tone through image analysis weekly
Improves the appearance of dark spots through image analysis weekly
Improves the signs of aging through image analysis weekly
Reduced the appearance of melasma through image analysis weekly
Helped stimulate collagen through image analysis weekly
Diminished the appearance of acne and blackheads through image analysis weekly
Decreases the appearance of pores and texture through image analysis weekly
Improves overall tolerance level assessed by clinical grading and participant reporting
Improves confidence in overall skin assessed by participant reporting

### Study Design

The study had followed the protocols set forth in Table 5.

TABLE 5

| | |
|---|---|
| Week 1 | Formulation 1 (Hexylrescorinol pads) |
| Week 2 | Formulation 2 (Butylresorcinol pads) |
| Week 3 | Formulation 3 (Phenylethyl resorcinol pads) |
| Week 4 | Formulation 4 (Blend resorcinol pads) |
| Week 5 | Formulation 5 (varied dosage of actives in |
| | blend resorcinol pads) |

### Evaluation Procedures

All evaluations were conducted in accordance with the following scales and procedures. Assessment of efficacy was based on brightness, pigmentation, skin tone, dark spots, pores and texture. To evaluate the brightness, a trained technician evaluated skin brightness on the face of each subject according to the scale below.

Scale for Skin Brightness: 0=No dullness present/bright complexion; 1-3=slight dull appearance; 4-6=moderate dull appearance; 7-9=severe dull appearance.

To evaluate the pigmentation, a trained technician assessed the photograph and evaluate pigmentation on the face of each subject according to the scale below.

Scale for Scoring Skin Pigmentation: 0=No discoloration present; 1-3=slight discoloration visible; 4-6=moderate discoloration visible; 7-9=severe discoloration visible.

To evaluate the skin tone, a trained technician assessed the photograph and evaluated the skin tone on the face of each subject according to the scale below.

Scale for Scoring Skin tone: 0=even skin tone; 1-3=slight areas of uneven skin tone; 4-6=moderate areas of uneven skin tone; 7-9=severe areas of uneven skin tone.

To evaluate the dark spots, a trained technician assessed the photograph and evaluated the dark spots on the face of each subject according to the scale below.

Scale for Scoring Dark Spots: 0=no discoloration present; 1-3=slight discoloration visible; 4-6=moderate discoloration visible; 7-9=severe discoloration visible.

To evaluate the tolerability, a trained technician assessed the photograph and evaluated the skin on the face of each subject according to the scale below.

| | 0 | 1 | 2 | 3 |
|---|---|---|---|---|
| Erythema (redness) | ◯ | ◯ | ◯ | ◯ |
| Edema (swelling) | ◯ | ◯ | ◯ | ◯ |
| Dryness | ◯ | ◯ | ◯ | ◯ |
| Burning | ◯ | ◯ | ◯ | ◯ |
| Stinging | ◯ | ◯ | ◯ | ◯ |
| Itching | ◯ | ◯ | ◯ | ◯ |
| Peeling | ◯ | ◯ | ◯ | ◯ |

Tolerability Assessment *
0 = None, 1 = mild, 2 = moderate, 3 = severe

At the end of each week, a photo was taken of the subjects face from the front, right and left views. In order to ensure consistency between the photographs, each subject was asked to were a black shirt and black headband to pull hair off and away from the face. The images were analyzed to determine changes in the appearance of brightness, pigmentation, skin tone, dark spot, pores and texture.

All subjects were asked to abide the following: no makeup on; no mirror selfie; 30 degree angle—show the whole face and zoom out; hair out of face and tucked behind ears—in a black headband; remove distractors—jewelry, hair, glasses; eyes open looking directly at camera; keep mouth closed; subjects to wear a natural colored shirt (black or white); and good natural lighting.

At the end of each week, the subjects were asked to fill out a questionnaire.

### Results of Study

After 1 week of treatment with formulation 1, 0% had reported no improvement, 14.3% had reported slight improvement, 64.3% reported moderate improvement, and 21.4% had reported significant improvement. The subjects were also asked to report on sensitivity and it was found that 57.1% had no sensitivity, 28.6% had mild sensitivity, 7.1% had moderate sensitivity and 7.1% had severe sensitivity.

After 1 week of treatment with pads having formulation 2, 0% had reported no improvement, 14.3% had slight improvement, 57.1% had moderate improvement and 28.6% had significant improvement. Regarding sensitivity, 57.1% had no sensitivity, 21.4% had mild sensitivity, 21.4% had moderate sensitivity and 0% had severe sensitivity.

After 1 week of treatment with pads having formulation 3, 0% had reported no improvement, 20% had slight improvement, 20% had moderate improvement and 60% had significant improvement. Regarding sensitivity, 80% had no sensitivity, 20% had mild sensitivity, 0% had moderate sensitivity and 0% had severe sensitivity.

After 1 week of treatment with pads having formulation 4, 0% had reported no improvement, 0% had slight improvement, 77.8% had moderate improvement and 22.2% had significant improvement. Regarding sensitivity, 66.7% had no sensitivity, 33.3% had mild sensitivity, 0% had moderate sensitivity and 0% had severe sensitivity.

After 1 week of treatment with pads having formulation 5, 0% had reported no improvement, 0% had slight improvement, 44.4% had moderate improvement and 55.6% had significant improvement. Regarding sensitivity, 88.9% had no sensitivity, 11.1% had mild sensitivity, 0% had moderate sensitivity and 0% had severe sensitivity.

Thus, the present study supports using soluble resorcinol derivatives as well as the delivery system result in a very effective treatment for pigmentation that shows fast and better results than previous koji formulations. Through this study, it was noted that these pads are far less irritating, compliant with CARES Act, do not stain blackheads and increases efficacy of lightening pigmentation.

### Additional Consumer Study

A 12 week study was conducted to evaluate the efficacy and tolerability of a formulation according to the present disclosure to improve the appearance of skin pigment, brightness, pores, texture and tone. The study included 62 healthy subjects (60 female and 2 male) seeking treatment of pigmentation or related disorders. The subjects were aged 24 to 70 years having a median age of 35 years and Fitzpatrick skin types I (1.6%), II (20.6%), III (49.2%), IV (23.8%), and V (4.8%).

The subjects agreed to follow a skin care regimen with test products provided (and none other) and to avoid excessive sun exposure and tanning beds during the study period. The subjects were excluded if they took oral or topical medications known to cause of affect pigmentation (e.g., hydroquinone) or had large amounts of facial hair, neck or facial tattoos or piercings, visible skin disease, allergies to ingredients of the test products, or a major illness.

The subjects received a skin evaluation at the baseline visit. The subjects were given the test product which included the formulation according to the present disclosure and instructions on its use. The formulation was a mixture of 4-hexylresorcinol, 4-n-butylresorcinol, phenyl-ethylresorcinol [4-(1-phenylethyl)1,3-benzene diol], arbutin, ascorbic acid, salicylic acid, and kojic acid. The subjects were photographed without makeup under standardized conditions of position, dress, background and lighting before and after the 12 weeks of treatment.

The subjects were instructed to use the test product at least 5 times weekly and to apply the test product up to twice daily if tolerated. The subjects were permitted to use the test product more than 5 times per week, but not more than twice daily during the study period. The subjects were also given cleanser, sunscreen SPF 50, and moisturizer from RFA Skincare in addition to their test products for a full well-rounded routine. The subjects were asked not to use any new skincare products during this study and were given a diary to record changes in their complexion and adverse events. The subjects reported to the testing facility for evaluation and photography after 6 and 12 weeks of using the test product. The subjects were permitted to miss no more than two non-consecutive weeks of self-treatment during the study period.

The subjects were instructed to use the products in the morning and again in the evening. The subjects were told to apply the test produce over the face and neck, using caution to avoid the eyes, in the evening.

Efficacy: At baseline and 12 weeks, subjects evaluated facial skin pigmentation, brightness/radiance, pores/texture, tone, and dark spots from photographs. Scores were on a scale of 1 to 5 where 1, for example, denoted minimal skin brightness and 5 indicated excellent skin brightness. At 12 weeks, subjects also evaluated from photographs improvement in skin evenness, glow, lightening, and tightness on the scale 0=no improvement, 1=slight improvement, 2=moderate improvement, and 3=significant improvement. Finally, subjects graded overall satisfaction with the test product results on a 0 to 10 scale where 0=minimal satisfaction and 10=maximum satisfaction.

Tolerability: At 12 weeks, subjects evaluated erythema, edema, dryness, burning, stinging, itching, and peeling, using a scale of 0=none, 1=mild, 2=moderate, and 3=severe. At 12 weeks, overall irritation was evaluated using a scale of 0=no irritation, 1=mild irritation, 2=moderate irritation, 3=marked irritation, and 4=severe irritation.

Diaries: The subjects kept diaries where they commented on the signs of aging (fine lines, wrinkles, dull skin, enlarged pores), collagen stimulation (plumpness of skin), acne, blackheads (comedomes with opened pores), whiteheads (comedones with closed pores), melasma (in 31 subjects), pigmentation, sun damage, sun spots, and age spots while using the test product. The subjects also comments on confidence in their skin without wearing makeup while using the test product.

Statistics: Because the scores were small, ordinal whole numbers, nonparametric statistics were used to evaluate the results. For the entire group of 62 subjects, efficacy (brightness/radiance, pores/texture, skin tone, dark spots) at baseline was compared to final value at 12 weeks using the paired Wilcoxon test using p<0.05 as the cutoff for significance. Efficacy, adverse effects, and improvement data were divided into groups according to Fitzpatrick skin type (types I and II, type III, and types IV and V) and age (24 to 32, 33 to 38, and 39 to 70 years of age). The efficacy, adverse effects, and improvement scores of the three skin type groups were compared with the Kruskal Wallis test to determine if these parameters differed significantly among the three groups. The same was done with the three age groups. In both cases the cutoff level for significance was p<0.05.

### Results

The results are shown in Tables 1, 2 and 3. Table 1 shows that differences between the baseline and 12 weeks for the entire group of subjects were statistically significant (p<0.0001) for each efficacy parameter. Median values at 12 weeks were 4.0 to 5.0 (good to excellent in each case.

TABLE 1
Comparison of efficacy between baseline
and 12 weeks by Wilcoxon test

| | Median (IQR) | | |
|---|---|---|---|
| Efficacy parameter | Baseline | 12 weeks | P value |
| Brightness/radiance | 3.0 (1.0) | 5.0 (1.0) | <0.0001 (s) |
| Pores/texture | 2.0 (1.0) | 4.0 (2.0) | <0.0001 (s) |
| Skin tone | 3.0 (1.0) | 4.0 (1.0) | <0.0001 (s) |
| Dark spots | 2.0 (1.0) | 4.0 (1.0) | <0.0001 (s) |

IQR = Interquartile range = 3^{rd} quartile-1^{st} quartile; s = significant.

Table 2 shows the median values for efficacy, adverse effects, and improvement in skin parameters for the Fitzpatrick skin types. In each case the differences among the groups were not significant, indicating that effects of the test product were consistent among the three Fitzpatrick skin type groups. Table 3 shows similar results for the three age groups as well as overall satisfaction.

TABLE 2
Efficacy (12 wk-baseline), adverse effects (12 wk),
improvement (12 wk) and overall satisfaction (12
wk) by Fitzpatrick skin type (median, IQR)

| Skin | Fitzpatrick skin type | | | |
|---|---|---|---|---|
| parameter | I-II | III | IV-V | P value |
| Efficacy | | | | |
| Brightness/ | 2.0 (1.0) | 2.0 (2.0) | 2.0 (1.1) | 0.2986 (ns) |
| radiance | | | | |
| Pores/texture | 1.5 (2.0) | 2.0 (1.0) | 1.0 (1.2) | 0.7283 (ns) |
| Tone | 1.5 (1.1) | 2.0 (1.1) | 1.0 (2.0) | 0.5091 (ns) |
| Dark spots | 1.5 (1.2) | 2.0 (2.0) | 1.0 (1.1) | 0.7742 (ns) |
| Adverse | | | | |
| Effects | | | | |
| Erythema | 1.0 (1.0) | 0.0 (1.0) | 0.0 (1.0) | 0.2113 (ns) |
| Edema | 0.0 (0.0) | 0.0 (0.0) | 0.0 (0.0) | 0.2946 (ns) |
| Dryness | 1.0 (0.1) | 1.0 (1.0) | 1.0 (1.0) | 0.5664 (ns) |
| Burning | 0.0 (0.1) | 0.0 (0.1) | 0.0 (1.0) | 0.9078 (ns) |
| Stinging | 0.0 (1.0) | 0.0 (0.1) | 0.0 (0.0) | 0.4766 (ns) |
| Itching | 0.0 (0.0) | 0.0 (0.0) | 0.0 (0.1) | 0.0934 (ns) |
| Peeling | 0.0 (0.0) | 0.0 (0.0) | 0.0 (0.0) | 0.9736 (ns) |
| Overall | 0.5 (1.0) | 0.0 (1.0) | 0.0 (1.0) | 0.8285 (ns) |
| irritation | | | | |
| Improvement | | | | |
| Evenness | 3.0 (1.0) | 3.0 (0.0) | 3.0 (1.0) | 0.2467 (ns) |
| Glow | 3.0 (0.0) | 3.0 (0.0) | 3.0 (0.0) | 0.2882 (ns) |
| Lightening | 3.0 (1.0) | 3.0 (1.0) | 3.0 (1.0) | 0.4952 (ns) |
| dark spots | | | | |
| Tightening | 2.5 (1.0) | 3.0 (1.0) | 2.0 (1.0) | 0.4044 (ns) |
| Overall | 10.0 (0.1) | 10.0 (0.1) | 10.0 (1.0) | 0.6016 (ns) |
| satisfaction* | | | | |

IQR—interquartile range (3rd quartile-1^{st} quartile);
ns = nonsignificant
*Scale 1 to 10.

TABLE 3
Efficacy (12 wk-baseline), adverse effects (12 wk), improvement
(12 wk) and overall satisfaction (12 wk) by age (median, IQR)

| Skin | Age (years) | | | |
|---|---|---|---|---|
| parameter | 24-32 | 33-38 | 39-70 | P value |
| Efficacy | | | | |
| Brightness/ | 2.0 (2.0) | 2.0 (1.1) | 2.0 (1.0) | 0.6492 (ns) |
| radiance | | | | |
| Pores/texture | 2.0 (1.3) | 1.0 (1.1) | 2.0 (1.0) | 0.1455 (ns) |
| Tone | 2.0 (2.0) | 1.5 (1.1) | 2.0 (1.0) | 0.8791 (ns) |
| Dark spots | 1.0 (1.6) | 2.0 (2.0) | 1.0 (1.0) | 0.4225 (ns) |
| Adverse | | | | |
| Effects | | | | |
| Erythema | 1.0 (1.0) | 0.5 (1.0) | 0.0 (1.0) | 0.5855 (ns) |
| Edema | 0.0 (0.0) | 0.0 (0.0) | 0.0 (0.0) | 0.4029 (ns) |
| Dryness | 1.0 (0.3) | 1.0 (1.0) | 1.0 (1.0) | 0.4062 (ns) |
| Burning | 0.0 (0.0) | 0.0 (1.0) | 0.0 (0.0) | 0.0639 (ns) |
| Stinging | 0.0 (0.3) | 0.0 (1.0) | 0.0 (0.0) | 0.9020 (ns) |
| Itching | 0.0 (0.0) | 0.0 (0.0) | 0.0 (0.0) | 0.6684 (ns) |
| Peeling | 0.0 (0.0) | 0.0 (0.0) | 0.0 (0.0) | 0.6688 (ns) |
| Overall | 0.0 (1.0) | 0.0 (1.0) | 0.0 (1.0) | 0.9537 (ns) |
| irritation | | | | |
| Improvement | | | | |
| Evenness | 3.0 (0.3) | 3.0 (0.0) | 3.0 (1.0) | 0.3069 (ns) |
| Glow | 3.0 (0.0) | 3.0 (0.0) | 3.0 (0.0) | 0.0464 (ns) |
| Lightening | 3.0 (1.0) | 3.0 (1.0) | 3.0 (1.0) | 0.8537 (ns) |
| dark spots | | | | |
| Tightening | 3.0 (1.0) | 2.0 (1.0) | 3.0 (1.0) | 0.4753 (ns) |
| Overall | 10.0 (1.0) | 10.0 (0.1) | 10.0 (0.8) | 0.9344 (ns) |
| satisfaction* | | | | |

IQR—interquartile range (3rd quartile-1^{st} quartile);
ns = nonsignificant
*Scale 1 to 10.

Table 4 shows comments noted by subjects regarding their use of the test product for 12 weeks. One hundred percent of subjects became more confident in the appearance of their skin without makeup and 95% would recommend the test product to friends. All subjects reported that results were better than those of other products they had used.

TABLE 4
Percentage of subjects that commented in diaries about
improvements in skin while using the test product

| Comment | Subjects (%) |
|---|---|
| Signs of aging | 59 |
| Stimulated collagen | 40 |
| Reduced acne (reduction in number, size, and severity of | 37 |
| facial breakouts) | |
| Reduced blackheads (acne) | 25 |
| Cleared melasma | 50 |
| More confident in skin appearance | 100 |
| Would recommend product to friends | 95 |
| Reduced acne-induced pigmentation | 44 |
| Rapid results | 70 |
| Reduced appearance of aging spots | 44 |
| Pleasant experience in using product | 94 |
| No greasy skin while using product | 90 |
| Improved appearance of sun damage | 75 |
| Improved appearance of sun spots | 65 |
| Skin not sticky or dry while using product | 86 |
| Results better than those of other products | 84 |

The results show that the test product provided subject-noted improvement in skin brightness/radiance, pores and texture, tone, and dark spots with minimal adverse effects. The subjects also reported improved skin evenness, glow, tightness, and lightened dark spots. The subjects were more confident in the appearance of their skin without makeup, 95% would recommend the test product to friends, 84% reported that results were better than those of other products they had used, and 100% of subjects noticed a transformation of their skin after using the test product.

The present study describes a new combination of agents that improved the appearance of skin brightness, pores and texture, tone, dark spots, tightness, evenness, and glow with minimal erythema, edema, dryness, burning, stinging, itching, peeling, and overall irritation. The subjects unanimously agreed that results with this combination were better than those of other products they had used and most would recommend it. These results suggest that the formulation of the present disclosure provides visible improvement in a short time, thus improving patient compliance and satisfaction. The strengths of the present study include the high number of subjects and use of a new combination of agents for treating hyperpigmentation. The limitations are the small number of Fitzpatrick skin type I subjects (n=1) and type V (n=3) subjects.

For both Fitzpatrick skin types and age groups, use of the combination of 4-hexylresorcinol, 4-n-butylresorcinol, phenyl ethylrescorcinol, arbutin, ascorbic acid, salicylic acid, and kojic acid provided consistent improvement in hyperpigmentation with minimal adverse effects for 12 weeks.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The term “about”, when referring to a physical quantity, is to be understood to include measurement errors within, and inclusive of 10%. For example, “about 100° C.” should be understood to mean “100±10° C.”.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A topical formulation comprising:
a tyrosine inhibitor, a skin brightener, and a resorcinol compound.

2. The topical formulation of claim 1, wherein the tyrosine inhibitor comprises kojic acid.

3. The topical formulation of claim 1, wherein the skin brightener comprises a glycosylated derivative of hydroquinone.

4. The topical formulation of claim 3, wherein the glycosylated derivative of hydroquinone comprises arbutin.

5. The topical formulation of claim 1, wherein the resorcinol compound comprises an alkyl resorcinol, an aromatic resorcinol or a combination thereof.

6. (canceled)

7. (canceled)

8. The topical formulation of claim 1, wherein the resorcinol compound comprises hexyl resorcinol, butyl resorcinol, phenylethyl resorcinol, or a combination thereof.

9. The topical formulation of claim 1, wherein the formulation does not include hydroquinone.

10. The topical formulation of claim 1, wherein the tyrosine inhibitor is included in an amount of about 0.5 wt % to about 10 wt % based on total weight of the formulation.

11. (canceled)

12. The topical formulation of claim 1, wherein the glycosylated derivative of hydroquinone is included in an amount of about 1 wt % to about 10 wt % based on total weight of the formulation.

13. (canceled)

14. The topical formulation of claim 1, wherein the resorcinol compound comprises hexyl resorcinol in an amount of about 0.1 wt % to about 10 wt %, based on total weight of the formulation.

15. The topical formulation of claim 1, wherein the resorcinol compound comprises butyl resorcinol in an amount of 0.1 wt % to about 10 wt %, based on total weight of the formulation.

16. The topical formulation of claim 1, wherein the resorcinol compound comprises phenylethyl resorcinol in an amount of about 0.01 wt % to about 10 wt %, based on total weight of the formulation.

17. The topical formulation of claim 1, wherein the resorcinol compound comprises hexyl resorcinol in an amount of about 0.1 wt % to about 10 wt %, butyl resorcinol in an amount of about 0.1 wt % to about 10 wt %, and phenylethyl resorcinol in an amount of about 0.01 wt % to about 10 wt %, based on total weight of the formulation.

18. The topical formulation of claim 1, further comprising an ascorbate component.

19.-34. (canceled)

35. The topical formulation of claim 1, further comprising a keratolytic.

36.-39. (canceled)

40. The topical formulation of claim 1, further comprising an emulsifier.

41.-44. (canceled)

45. The topical formulation of claim 1, further comprising potassium hydroxide.

46. (canceled)

47. The topical formulation of claim 1, further comprising a chelating agent.

48.-64. (canceled)

65. A delivery system comprising an applicator pad infused with a topical formulation according to claim 1.

66. A method of treating skin comprising applying a topical formulation according to claim 1 to the skin of a patient.

67.-71. (canceled)
